(19) United States
(12) Plant Patent Application Publication
(10) Pub. No.: US 2004/0128723 P1
(43) Pub. Date: Jul. 1, 2004

(54) DIASCIA PLANT NAMED 'BALWINLOT'

(75) Inventor: Scott C. Trees, Arroyo Grande, CA (US)

Correspondence Address: WOOD, PHILLIPS, KATZ, CLARK & MORTIMER, 500 W. MADISON STREET, SUITE 3800, CHICAGO, IL, 60661, US

(73) Assignee: Ball FloraPlant, a division of Ball Horticultural Company

(21) Appl. No.: 10/329,589

(22) Filed: Dec. 26, 2002

(51) Int. Cl. A01H 5/00

(52) U.S. Cl. PLT/263

(57) ABSTRACT

A new and distinct Diascia plant named ‘Balwinlot’, characterized by its light pink colored flowers, spreading, trailing growth habit, and medium green foliage.

### LATIN NAME OF GENUS AND SPECIES OF PLANT CLAIMED

Diascia barberae

### VARIETY DENOMINATION

‘Balwinlot’

### BACKGROUND OF INVENTION

The present invention comprises a new and distinct Diascia plant, botanically known as Diascia barberae, and hereinafter referred to by the cultivar name ‘Balwinlot’.

The new cultivar was developed by the inventor in a controlled breeding program during October 2000, at Arroyo Grande, Calif. The objective of the breeding program was the development of Diascia cultivars with a well-branched, spreading growth habit, continuous flowering and medium green foliage.

The new cultivar was the result of open pollination with the female parent being a proprietary Diascia selection designated MH-026. ‘Balwinlot’ was discovered and selected in January 2001 as a single flowering plant from within the progeny of the above stated cross and was designated DIAS-150.

Asexual reproduction of the new cultivar at West Chicago, Ill. was carried out by the use of terminal stem cuttings and has demonstrated that the characteristics of the new cultivar as herein described are firmly fixed and retained through successive generations of such asexual propagation.

### SUMMARY OF INVENTION

It was found that the cultivar of the present invention:

(a) exhibits light pink colored flowers,

(b) forms foliage of a medium green color, and

(c) exhibits a spreading and trailing growth habit.

The new cultivar can be compared to the commercially available Diascia variety, Sun Chimes™ ‘Peach’ (nonpatented). In side-by-side comparison, plants of the new cultivar are taller and narrower, and have lighter pink flowers than those of Sun Chimes™ ‘Peach’.

### BRIEF DESCRIPTION OF PHOTOGRAPH

The accompanying photographs show as nearly true as it is reasonably possible to make the same in color illustrations of this type, typical flower and foliage characteristics of the new cultivar. The plants were grown for 11 weeks in a greenhouse at West Chicago, Ill.

FIG. 1 illustrates the overall growth habit of the new cultivar.

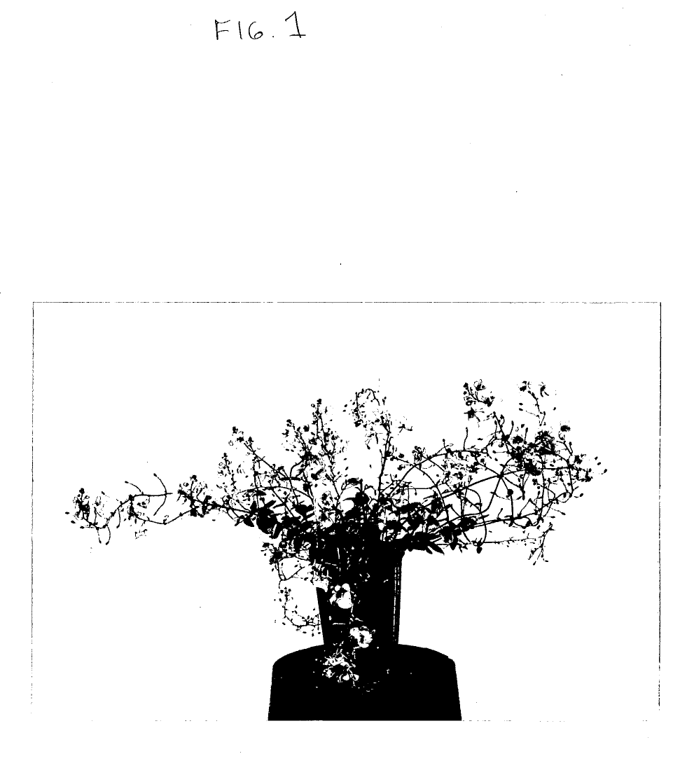

FIG. 2 illustrates a close up view of individual flowers of the new cultivar.

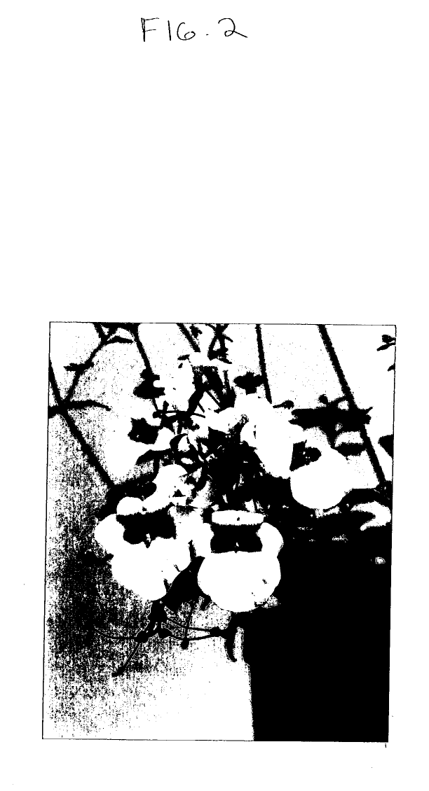

### DETAILED BOTANICAL DESCRIPTION

The new cultivar has not been observed under all possible environmental conditions to date. Accordingly, it is possible that the phenotype may vary somewhat with variations in the environment, such as temperature, light intensity, and day length. The chart used in the identification of colors described herein is the R.H.S. Colour Chart of The Royal Horticultural Society, London, England. The color values were determined on Apr. 24, 2002. The readings were taken between 1:00 and 3:00 p.m. under natural daylight conditions. The plants were produced from cuttings taken from stock plants and were grown in a double polycarbonate covered greenhouse under conditions comparable to those used in commercial practice. The plants were grown for 9 weeks while utilizing a soilless growth medium and maintaining temperatures of approximately 62° to 75° F. (17° to 24° C.) during the day and approximately 52° to 60° F. (11° to 15° C.) during the night and light levels of 5,000 to 8,000 footcandles.

Classification:

Botanical.—Diascia barberae cultivar ‘Balwinlot’.

Parentage:

Female parent.—Proprietary Diascia selection MH-026.

Propagation:

Type cutting.—Terminal tip.

Time to initiate roots.—Approximately 14 to 21 days.

Root description.—Fibrous, branching.

Plant description:

Crop time.—Approximately 5 to 7 weeks from planting of rooted cuttings in a 10 cm pot.

Habit of growth.—Vigorous. Pinching improves branching. A mature plant, 9 weeks after the planting of a rooted cutting, measures approximately 27.9 cm in height and approximately 37 cm in width with approximately 5.5 main branches and 21 lateral flowering branches.

Form.—Spreading, trailing.

Lateral branch.—Shape: Square. Length: Approximately 17.5 cm. Diameter: Approximately 1.4 mm. Texture: Moderately pubescent. Internode length at middle of branch: Approximately 2.6 cm. Color: 144A.

Foliage.—Type: Simple. Arrangement: Opposite. Shape: Ovate. Margin: Serrate. Apex: Acute. Base: Cordate. Texture: Glabrous, supple. Quantity per lateral branch: Approximately 9. Leaf length: Approximately 2.4 cm. Leaf width: Approximately 1.7 cm. Leaf orientation to stem: Acute. Leaf fragrance: None. Upper surface of mature foliage is 137C and lower surface is lighter than 137D. Upper and lower surfaces have palmate venation of 144A. Petiole length: Approximately 4 mm. Petiole diameter: Approximately 2 mm. Petiole color: 144A. Upper and lower surfaces of petiole are glabrous.

Flowering description:

Flowering habit.—Freely flowering.

Natural flowering season.—Year round in greenhouse environment and spring through autumn in outdoor garden.

Flower arrangement.—Terminal racemes.

Flower description:

Type.—Solitary, five lobed and zygomorphic.

Fragrance.—None.

Aspect.—Concave.

Corolla.—Width: Approximately 2 cm. Length: Approximately 2 cm. Depth: Approximately 5 mm.

Petals.—Number: Five, fused at base. Aspect: Cupped. Two upper petals: Length is approximately 5 mm, width is approximately 4 mm. Lateral petals: Length is approximately 5 mm, width is approximately 7 mm. sharply curved nectar spurs originate from base of lateral petals. Nectar spur length: Approximately 6.5 mm. Nectar spur color: 37D. Lower petal: Length is approximately 8 mm, width is approximately 1.1 cm. All petals: Margin: Entire. Apex: Obtuse. Texture: Glabrous and iridescent. Color: Upper surface of upper petal is closest to 37D with indentation at base of 1A. Lower surface of upper petal is 37D. Upper surface of lateral petals is 37C. Lower surface of lateral petals is 37D. Upper surface of lower petal is 37C. Lower surface of lower petal is 37D.

Sepals.—Quantity: 5. Shape: Lanceolate. Length: Approximately 3 mm. Width: Approximately 1 mm. Apex: Acute. Margin: Entire. Calyx shape: Star. Calyx length: Approximately 5 mm. Calyx diameter: Approximately 6 mm. Calyx texture: Densely pubescent with glands of 93A on outer surface. Inner surface is glabrous. Color of outer surface is 143B. Color of inner surface is 143D.

Pedicel.—Length: Approximately 9 mm. Width: Less than 1 mm. Strength: Moderate. Texture: Moderately pubescent. Color: 144A.

Peduncle.—Length: Approximately 5.8 cm. Diameter: Approximately 1.3 mm. Aspect: At acute angle to branch. Texture: Sparsely pubescent. Color: 144A.

Bud.—Shape: Oval, flat. Length: Approximately 4.6 mm in length. Diameter: Approximately 6.4 mm. Color: Between 37B and 37C.

Reproductive organs.—Androecium: There are 4 stamens. Anther shape: Oval. Anther length: Approximately 1 mm. Anther color: 1C. Pollen amount: Abundant. Pollen color: 13B. Gynoecium: There is one pistil, 4 mm in length. Stigma shape: Round. Stigma length: Less than 1 mm. Stigma color: 150C, transparent. The style length is approximately 1.5 mm and the color is 154D. Ovary length is approximately 1.5 mm and color is N144C.

Seed production: Seed production has not been observed.

Disease resistance: Resistance to pathogens has not been observed.

Hardiness zone: ‘Balwinlot’ is hardy in zones nine (9) and above.

I claim:

1. A new and distinct Diascia barberae plant named ‘Balwinlot’ substantially as herein shown and described, which:
(a) exhibits light pink colored flowers,
(b) forms medium green foliage, and
(c) exhibits a spreading and trailing growth habit.
